(19) United States
(12) Patent Application Publication
(10) Pub. No.: US 2015/0247844 A1
(43) Pub. Date: Sep. 3, 2015

(54) Method for Obtaining Cell and Tissue Specific Biomolecules

(71) Applicants: Stanislav L. Karsten, Signal Hill, CA (US); Lili C. Kudo, Signal Hill, CA (US)

(72) Inventors: Stanislav L. Karsten, Signal Hill, CA (US); Lili C. Kudo, Signal Hill, CA (US)

(21) Appl. No.: 14/194,456

(22) Filed: Feb. 28, 2014

(51) Int. Cl. G01N 33/543; C12Q 1/68; G01N 1/30

(52) U.S. Cl. CPC G01N 33/54333; G01N 1/30; C12Q 1/6806

(57) ABSTRACT

Methods for tissue microdissection- or dissociation-free means of collecting cell-specific biomolecules from complex heterogeneous tissue sources including native tissues, fresh frozen, fixed, and archived specimens. The tissue section having a plurality of beads with immobilized capture probes on their surface evenly distributed over all cell types of the tissue is arranged. Alternatively the tissue section being coated with a thin layer of resin containing incorporated or covalently bound immobilizing capture probes is arranged. The beads or resin are incubated directly on the tissue section permitting capturing of the biomolecules by the capture probes. After incubation, the tissue section is visualized and the cells or area of interest are identified. Next, the beads or resin samples, located in these cells or areas of interest, are collected and transferred to a sample tube where biomolecules are separated from the beads or resin using standard methods and are then used in downstream applications.

### RELATED APPLICATIONS

Not applicable.

### INCORPORATION BY REFERENCE

Applicant(s) hereby incorporate herein by reference any and all patents and published patent applications cited or referred to in this application.

### BACKGROUND OF THE INVENTION

1. Field of the Invention:

Aspects of this invention relate generally to biomaterial collection, and more particularly to compositions and methods for obtaining cell and tissue samples.

2. Description of Related Art:

The field of the invention is biomaterial collection, more specifically, the present invention relates to direct collection of cell and region specific biomolecules including DNA, RNA, and proteins as an alternative to tissue microdissection and flow sorting. The biomaterial collection compositions and methods presented herein can be used in conjunction with the capillary-based cell and tissue acquisition system (“CTAS”) as disclosed in WO/2008/021202, which is incorporated herein by reference in its entirety.

All referenced patents, applications and literature are incorporated herein by reference in their entirety. Furthermore, where a definition or use of a term in a reference, which is incorporated by reference herein, is inconsistent or contrary to the definition of that term provided herein, the definition of that term provided herein applies and the definition of that term in the reference does not apply. The invention may seek to satisfy one or more of the above-mentioned desires. Although the present invention may obviate one or more of the above-mentioned desires, it should be understood that some aspects of the invention might not necessarily obviate them.

The following summary describes aspects of the present state of this field:

Cell specific analysis is of critical importance in clinical diagnostics, drug discovery, molecular studies of complex multicellular organisms, and the practice of medicine. Most diseases affect specific cell types, therefore analysis of individual cells, groups of cells, or subanatomical parts affected by the disease is a prerequisite for efficient diagnostic and drug development process. The mixtures of different cell types result in “masking effect” hiding cell or region specific molecular information. This is a central issue in neuroscience where brain tissues reveal incredible complexity and a disease usually affects only specific subanatomical brain regions, cells, or cell types, challenging our understanding of basic brain functions and complicating the process of drug discovery or diagnostics. Therefore, to date, availability of cell and tissue specific molecular information is incremental for sound molecular studies leading to the identification of novel drug targets, biomarkers, and pathways underlying disease pathogenesis.

However, procurement of pure cell populations or specific areas from native, fresh and archived tissue samples in order to obtain cell- and region specific molecular information, such as gene expression profile, is a challenging task requiring sophisticated equipment.

Current technologies for acquisition of cell specific molecular data are mainly represented by laser assisted microdissection instruments and fluorescence assisted cell sorting (“FACS”) systems. Both approaches involve physical separation or dissociation of the specific cells or regions from the heterogeneous complex tissue. Once collected, the cells may be used for the isolation of the desired biomolecules such as RNA or proteins for their use in the array of downstream applications (e.g. gene expression profiling, sequencing, proteomics, etc.).

There are several microdissection techniques ranging from manual microdissection to as mentioned above laser-assisted technologies, including laser ablation, laser pressure catapulting, laser capture microdissection, and micropunching or aspiration. Recently, a capillary-based vacuum-assisted cell and tissue acquisition system (“CTAS”) was developed based on WO/2008/021202 (Kudo et al., 2012).

Manual tissue dissection is usually performed using 5- to 100-μm-thick sections placed on non-coated glass slides. It is labor-intensive, time-consuming, operator dependent, and has a high risk of contamination. Moreover, manual microdissection is limited to the collection of fairly large anatomical and subanatomical tissue regions and does not permit cellular resolution. Laser-assisted microdissection techniques are significantly automated, increasing cell and tissue acquisition time, reducing contamination, and increasing overall experimental reproducibility. Perhaps the most common laser assisted microdissection technology is a laser capture microdissecting (“LCM”) that has been widely used in the past decades. Laser-assisted microdissection is performed on post-processed or fixed, including archived, tissues. It often involves heat and irradiation processes (e.g., laser ablation or UV laser cutting), affecting the quality of macromolecules, such as RNA and proteins, that increases the risk of experimental variability and artifact generation. For example, prerequisite tissue processing prior to LCM may decrease the RNA quality by more than 30%.

Fluorescence assisted cell sorting instruments are capable of separating a heterogeneous suspension of cells into purified fractions on the basis of fluorescence and light scattering properties. Briefly, the cells or particles to be analyzed are placed in suspension and injected into a directed fluid stream. This stream containing particles sequentially intersects one or more laser beams placed orthogonal to the flow. The laser beams are focused such that they only illuminate a single particle at any given time. If the given cell or particle contains a fluorescent tag excited by the laser, it will fluoresce, and the signals are collected by photodetectors, processed by specialized electronics, and stored on a computer. Cells with the specific signals can be directed in the collection tube and used for further analysis. At constant laser power, the intensity of emission will be dependent on the number of fluorophores present, thereby making flow cytometry both a qualitative and highly quantitative analysis tool. Some drawbacks of this technology are the invasive nature of tissue dissociation resulting in possible artifacts and the requirement of a special fluorescent label that might not always be readily available.

Both laser-assisted microdissection instruments and flow sorting machines are usually very expensive, limiting accessibility to these technologies for many research groups.

Sliced tissue samples are used for immunohistochemistry and in situ hybridization methods to label and detect specific protein and RNA populations using antibodies and RNA/DNA probes, respectively. The sliced surfaces of the tissue samples include cells that have been cut open, and offer accessibility to the biomolecules within the cells. Further treatment with agents, such as Triton X, can also permeabilize cells on the tissue slice, allowing probes to enter the cells and attach to the specific targets. However, such methods render the tissue slices unusable for further investigation or other experimentation. Current invention utilizes either capturing probe coated beads or resin also containing capturing probes (such as, but not limited to, oligonucleotides (oligo-dT) linked to agarose, Sigma). Briefly, direct biomolecule acquisition from desired cells and regions of heterogeneous tissue is accomplished by evenly coating a tissue section with magnetic beads or resin (e.g., sepharose, agarose) harboring biomolecule capture molecules (e.g., oligo-dT or specific antibodies), and then collecting the beads or resin samples from the desired regions of interest. Biomolecules such as RNA, DNA, or proteins captured by the probes attached to the beads or resin can be used for downstream application including proteomics or transcriptomics. In the current invention, by collecting the beads or resins with attached probes from the sample, the tissue slice is minimally damaged and morphologically intact and thus can undergo another treatment with a different set of beads/resin, immunohistochemistry, or in situ hybridization.

Magnetic beads with diameters from 1 micrometer and larger have been used in research settings for a number of years for the isolation of DNA, RNA, and proteins. Paramagnetic beads, spherical in shape, coated with probes for the desired biomolecule are applied to homogenized samples to capture the desired biomolecules then washed in order to remove the beads from the isolated sample. Inducible magnetic needle has been introduced in publication that allows collecting small magnetic beads in the order of micrometers in diameter.

Cellulose resins such as agarose or sepharose with covalently attached capture probes such as oligonucleotides, including but not limited to oligo-dT or poly-U, are used. Depending on the specific probe used, both beads and resins are capable of binding specifically various kinds of biomolecules. Region specific resin samples are collected using aspiration or recently developed CTAS (Kudo et al., 2012). Collected resin samples are transferred to the sample tube and captured biomolecules such as RNA are eluted and used for downstream applications.

Currently there is no known method or application that collects biomolecules from tissue and cell samples without dissection or homogenization.

Aspects of the present invention fulfill these needs and provide further related advantages as described in the following summary.

### SUMMARY OF THE INVENTION

Aspects of the present invention teach certain benefits in construction and use which give rise to the exemplary advantages described below.

Currently, isolation of cell-specific biomolecules involves either tissue microdissection performed by various microdissection devices such as laser capture microdissection or tissue dissociation or homogenization and subsequent sorting of the desired cells performed by flow sorting machines. This leads to the destruction of the original material being studied, which is not desirable when samples are rare or difficult to obtain. In addition, the procedure of biomolecule isolation from isolated cells has to be performed, which increases the time and cost of the cell- and tissue-specific studies. Aspects of the present invention relate to utilizing capturing beads or resins that allow the collection of biomolecules directly from tissue slices and cells, eliminating the steps of cell and tissue collection via dissection or sorting and without greatly affecting the tissue sections so that further experimentation will be possible on the exact same tissue slice or cells after the collection of beads. Such experimentation includes but is not limited to immunohisto/cytochemistry assay for a different biomolecule than the previous, in situ hybridization, etc.

An object of present invention is to provide tissue microdissection- or dissociation-free means of collecting cell-specific biomolecules from complex heterogeneous tissue sources including native tissues, fresh frozen, fixed, and archived specimens. In the present invention, the tissue section having a plurality of beads with immobilized capture probes on their surface evenly distributed over all cell types of the tissue or a layer of resin (such as, but not limited to, sepharose or agarose) with incorporated capture probes is arranged. The beads or resin are incubated directly on the tissue section permitting capturing of the biomolecules by the bead's or resin's capture probes. After incubation, the tissue section is visualized and the cells or area of interest are identified. In the next step, the beads or resin, located in these cells or areas of interest, are collected and transferred to the sample tube where biomolecules are separated from the beads or resin using standard methods and used in the downstream applications.

Cell-specific collection of beads or resin from the tissue section may be performed by a capillary-based vacuum-assisted cell and tissue acquisition system (“CTAS”; Kudo et al., 2012) operated at settings sufficient for the collection of the beads or resin samples but ensuring overall integrity of the tissue section as disclosed in WO/2008/021202.

Alternatively, other devices or methods now known or later developed for capturing beads or resin from a tissue section and for specifically collecting the beads or resin may be applied, such as but not limited to magnetic/electromagnetic force, forms of suction, material affinity, etc.

Glass, plastic, cellulose, and magnetic beads used for immobilization of biomaterials have relatively large variation in size and choice of immobilized probes which can be used as capturing compounds. For example, short oligo-dT probes may be used for direct capturing polyA RNA from the cells and tissues. Beads or resins with immobilized antibodies may be used for capturing of the specific proteins. Random oligonucleotide hexameres immobilized on the surface of the beads or covalently linked to the resins such as, but not limited to, agarose or sepharose may be used for capturing total RNA.

The method for even distribution of the beads or thin layer of the resin over the tissue section and cells according to aspects of the present invention may include the steps of tissue section immobilization on the glass slide and application of the solution containing the plurality of beads or a resin with immobilized capture probes on their surface with its further incubation in the buffer.

The apparatus for collection of beads or resin samples from tissue sections according to aspects of the present invention is provided with a chamber or a rod that has strong affinity for the beads or resin so as to hold the beads or resin and transfer them from the tissue section to a sample tube or other vessel where captured biomolecules may be removed from the beads or resin and used in downstream applications. An example of a rod with strong affinity for the beads or resin may be a magnetic rod efficiently aggregating magnetic beads on its surface.

According to aspects of the present invention, a single bead can be captured from among the plurality of beads distributed over the tissue surface. The beads from a single cell, group(s) of cells, or tissue area(s) may be collected, and biomolecules captured by the probes immobilized on the bead surface may be extracted, removed, and used in downstream applications including but not limited to cDNA synthesis, protein studies, genomics and proteomics applications, methylation studies of DNA, etc.

According to further aspects of the present invention, localized resin samples can be extracted from among the resin layer evenly distributed over the tissue surface. The resin from a single cell, group(s) of cells, or tissue area(s) may be collected, and biomolecules captured by the probes immobilized on the resin may be extracted, removed, and used in downstream applications including but not limited to cDNA synthesis, protein studies, genomics and proteomics applications, methylation studies of DNA, etc.

Other features and advantages of aspects of the present invention will become apparent from the following more detailed description, taken in conjunction with the accompanying drawings, which illustrate, by way of example, the principles of aspects of the invention.

### BRIEF DESCRIPTION OF THE DRAWINGS

The accompanying drawings illustrate aspects of the present invention. In such drawings:

FIG. 1A is a schematic view of a representative complex tissue section, in accordance with at least one embodiment;

FIG. 1B is a schematic view of bead application to the tissue section of FIG. 1A, including a side schematic view thereof, in accordance with at least one embodiment;

FIG. 1C is a schematic view of bead collection from the tissue section of FIG. 1B, including a side schematic view thereof, in accordance with at least one embodiment;

FIG. 1D is a side schematic view of an alternative exemplary embodiment of magnetic bead application to a further representative complex tissue section, in accordance with at least one embodiment;

FIG. 2 is a photo of a further alternative exemplary embodiment of bead application to a further representative complex tissue section, in accordance with at least one embodiment;

FIG. 3 is a photo of representative collected beads within a glass capillary as shown schematically in FIG. 1C, in accordance with at least one embodiment;

FIGS. 4A-4C are photos of representative collected resin samples from the thin layer of resin (1% agarose in this case) acquired with CTAS using capillaries of various diameter;

FIGS. 5A-5B are photos of representative collected resin samples (1% agarose in this case) within a glass capillary as shown schematically in FIG. 1C, in accordance with at least one embodiment; and

FIGS. 6A-6D are photos of further representative beads applied to and collected from the specific complex tissue areas as shown schematically in FIGS. 1B and 1C, in accordance with at least one embodiment.

The above described drawing figures illustrate aspects of the invention in at least one of its exemplary embodiments, which are further defined in detail in the following description. Features, elements, and aspects of the invention that are referenced by the same numerals in different figures represent the same, equivalent, or similar features, elements, or aspects, in accordance with one or more embodiments.

### DETAILED DESCRIPTION OF THE INVENTION

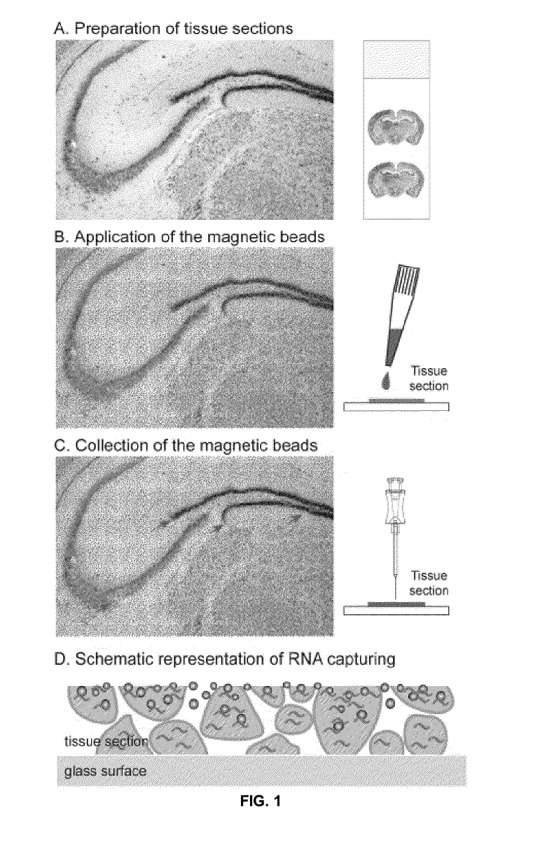

The above described drawing figures illustrate aspects of the invention in at least one of its exemplary embodiments, which are further defined in detail in the following description. Aspects of the invention and its biomaterial collection methods can now be better understood as set forth below in connection with FIGS. 1-5 disclosing exemplary method steps thereof. Turning now to FIG. 1, the general method steps in one embodiment are shown. First, in FIG. 1A there is shown a schematic view of a representative complex tissue section, in accordance with at least one embodiment; particularly, representing a step showing a tissue (a mouse brain in this particular example) prepared and sectioned. In FIG. 1B, there is shown the application of microbeads to the selected tissue section. Such beads may be magnetic, but not necessarily so; an example of a substantially non-magnetic bead that could be employed according to aspects of the present invention is oligo(dT)25-cellulose beads available from New England Biolabs (Catalog No. S1408S). The selected tissue section(s) is then incubated with the beads ensuring their near even distribution over the tissue section and incubated for a time period that is sufficient for beads to bind desired molecules. In FIG. 1C, there is shown the beads from the desired tissue region(s), group(s) of cells, or individual cells being collected and transferred into the test tube where captured biomolecules are released and used for the follow-up studies and the like. Alternatively, thin layer of resin with incorporated capture probes may be applied on the surface of the tissue instead of the beads (not shown). And in FIG. 1D, there is shown a schematic representation of RNA capturing comprising the beads applied to the tissue section on top of a glass surface such as a glass slide.

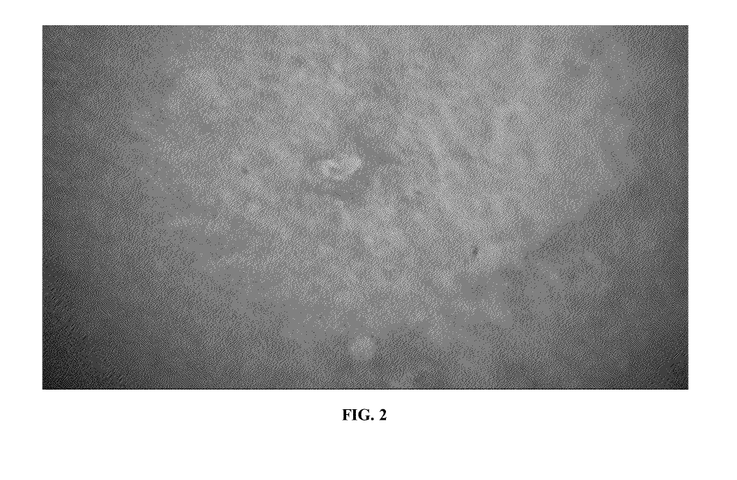

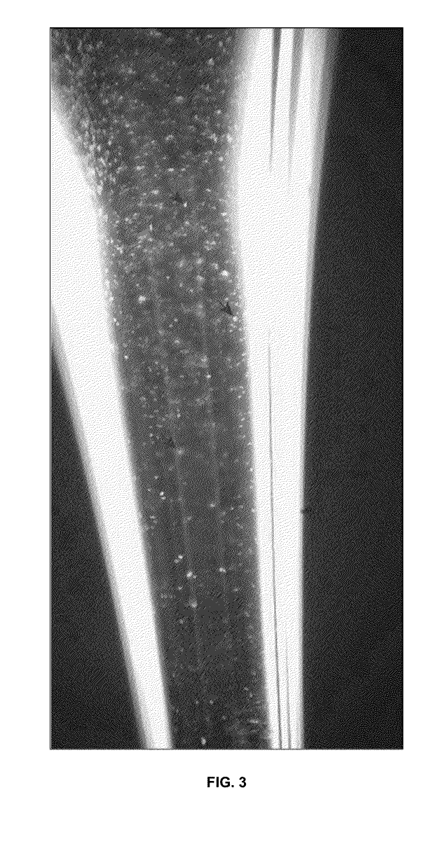

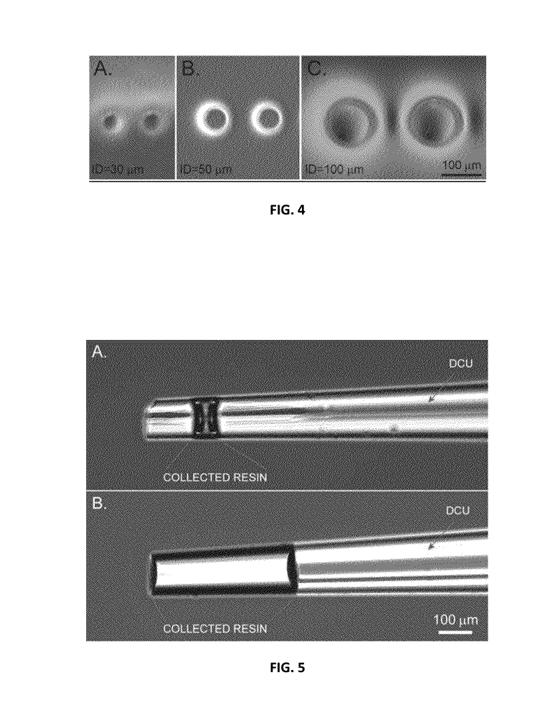

FIG. 2 is a photo of a further alternative exemplary embodiment of bead application to a representative complex tissue section, in accordance with at least one embodiment, here shown as a representative distribution of the beads over fresh frozen mouse brain tissue. FIG. 3 shows a representative embodiment of the collected beads (red arrows) in a glass capillary at four hundred times (400×) magnification level. FIGS. 4A-4C show a representative embodiment of the resin samples collected by CTAS using glass capillaries of various diameters (from 30 to 100 um). FIGS. 5A-5B show a representative embodiment of the resin samples (red) collected sequentially in a glass capillary again at four hundred times (400×) magnification level.

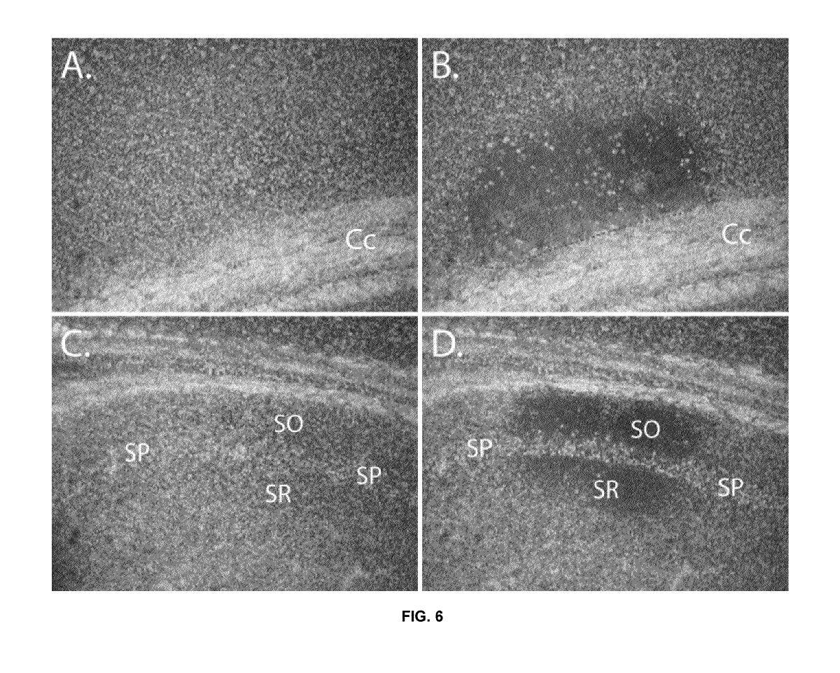

Finally, in FIGS. 6A-6D there are shown photos of further representative beads applied to and collected from complex tissue sections as shown schematically in FIGS. 1B and 1C, in accordance with at least one embodiment. In the alternative exemplary context, magnetic beads (0.5 mg/ml; dia.=1.0 μm; New England BioLabs) are shown directly applied to the tissue section (PFA-fixed mouse brain; 20 μm thickness) and collected from the desired areas without excessive tissue damage using CTAS v. 4.0. Particularly, in FIGS. 6A and 6B there are shown collection of beads from the 5^{th} and 6^{th} layers of the neocortex (Cc—corpus callosum). Representative beads (yellow) are indicated with red arrows and at four hundred times (400×) magnification once more. In FIGS. 6C and 6D there are shown the collection of beads from the stratum oriens (SO) and stratum radiatum (SR) regions of the hippocampal CA1 area (SP=stratum pyramidale). In the illustrated embodiments, beads were collected using borosilicate glass capillary (DCU i.d.=35 μm) after ten minutes (10 min) of incubation at RT (vacuum strength=6.6 in. Hg; pulse duration=0.2 sec).

To summarize, regarding the exemplary embodiments of the present invention as shown and described herein, it will be appreciated that methods for obtaining cell and tissue samples are disclosed. Because the principles of the invention may be practiced in a number of configurations beyond those shown and described, it is to be understood that the invention is not in any way limited by the exemplary embodiments, but is able to take numerous forms without departing from the spirit and scope of the invention. It should be noted that the various features of each above-described embodiment may be combined in any logical manner and are intended to be included within the scope of the present invention. It will also be appreciated by those skilled in the art that the present invention is not limited to the particular geometries and materials of construction disclosed, but may instead entail other functionally comparable structures or materials, now known or later developed, without departing from the spirit and scope of the invention.

Furthermore, while aspects of the invention have been described with reference to at least one exemplary embodiment, it is to be clearly understood by those skilled in the art that the invention is not limited thereto. Rather, the scope of the invention is to be interpreted only in conjunction with the appended claims and it is made clear here, that the inventors believe that the claimed subject matter is the invention.

What is claimed is:

1. A method of obtaining cell- and tissue-specific biomolecules, comprising the steps of:
preparing a selected tissue section;
arranging a means for sampling with immobilized capture probes on their surface substantially evenly distributed over the tissue section;
incubating the sampling means directly on the tissue section permitting capturing of the biomolecules by the capture probes;
identifying one or more areas of interest in the tissue section; and
collecting the sampling means located in the one or more areas of interest for biomolecule separation from the sampling means.

2. The method of claim 1 wherein the sampling means comprises a plurality of beads.

3. The method of claim 2 wherein the beads comprise magnetic beads having a nominal density of approximately 0.5 mg/ml and a nominal diameter of 0.1 to 1.0 μm.

4. The method of claim 2 wherein the step of arranging the beads over the tissue section comprises the steps of:
immobilizing the tissue section on a glass slide; and
placing the tissue section in a buffer solution containing the plurality of beads with immobilized capture probes on their surface.

5. The method of claim 2 wherein the step of collecting the beads from the tissue section comprises activating a magnetic rod having a strong affinity for the beads so as to hold the beads and transfer the beads from the tissue section to a sample tube.

6. The method of claim 2 wherein the step of collecting the beads from the tissue section comprises employing a capillary-based vacuum-assisted cell and tissue acquisition methodology.

7. The method of claim 1 wherein the sampling means comprises a thin layer of resin.

8. The method of claim 7 wherein the step of arranging the resin over the tissue section comprises the steps of:
immobilizing the tissue section on a glass slide; and
placing the tissue section in a buffer solution containing the thin layer of resin with immobilized capture probes on their surface.

9. The method of claim 7 wherein the step of collecting the resin samples from the tissue section comprises employing a capillary-based vacuum-assisted cell and tissue acquisition methodology.

10. The method of claim 1 wherein the step of preparing the selected tissue section comprises cutting the tissue to a thickness of approximately 5 to 500 μm.

11. The method of claim 1 wherein the capture probes are selected from the group consisting of short oligo-dT and poly-U probes, whereby polyA RNA may be collected from the tissue section.

12. The method of claim 1 wherein the capture probes comprise short random hexamer probes, whereby RNA or DNA may be collected from the tissue section.

13. The method of claim 1 wherein the capture probes comprise antibodies, whereby proteins may be collected from the tissue section.

14. A method of obtaining cell- and tissue-specific biomolecules, comprising the steps of:
preparing a selected tissue section;
arranging a plurality of magnetic beads with immobilized capture probes on their surface substantially evenly distributed over the tissue section, the step of arranging the beads comprising the further steps of:
immobilizing the tissue section on a glass slide; and
placing the tissue section in a buffer solution containing the plurality of beads with immobilized capture probes on their surface;
incubating the beads directly on the tissue section permitting capturing of the biomolecules by the capture probes;
identifying one or more areas of interest in the tissue section; and
collecting the beads located in the one or more areas of interest for biomolecule separation from the beads.

15. The method of claim 14 wherein the step of collecting the beads comprises activating a magnetic rod having a strong affinity for the beads so as to hold the beads and transfer the beads from the tissue section to a sample tube.

16. The method of claim 14 wherein the step of collecting the beads comprises employing a capillary-based vacuum-assisted cell and tissue acquisition methodology.

17. A method of obtaining cell- and tissue-specific biomolecules, comprising the steps of:
preparing a selected tissue section;
arranging a thin layer of resin with immobilized capture probes on their surface substantially evenly distributed over the tissue section, the step of arranging the resin comprising the further steps of:
immobilizing the tissue section on a glass slide; and
placing the tissue section in a buffer solution containing the thin layer of resin with immobilized capture probes on their surface;
incubating the resin directly on the tissue section permitting capturing of the biomolecules by the capture probes;
identifying one or more areas of interest in the tissue section; and
collecting the resin samples located in the one or more areas of interest for biomolecule separation from the resin.

18. The method of claim 17 wherein the step of collecting the resin samples from the tissue section comprises employing a capillary-based vacuum-assisted cell and tissue acquisition methodology.
